(19) United States
(12) Patent Application Publication
(10) Pub. No.: US 2011/0251461 A1
(43) Pub. Date: Oct. 13, 2011

(54) PROGRESSIVE SURGICAL DISTRACTION DEVICE FOR ATRAUMATIC ACCESS

(75) Inventors: Emilio Gómez González, Sevilla (ES); Guillermo Antiñolo Gil, Sevilla (ES); Javier Márquez Rivas, Sevilla (ES); Manuel A. Perales Esteve, Sevilla (ES)

(73) Assignee: Universidad De Sevilla, Sevilla (ES)

(21) Appl. No.: 13/061,282

(22) PCT Filed: Jul. 31, 2009
(86) PCT No.: PCT/ES09/70327

§ 371 Date: Jun. 3, 2011

(30) Foreign Application Priority Data
Aug. 1, 2008 (ES) P200802411

(51) Int. Cl. A61B 1/32

(52) U.S. Cl. 600/201

(57) ABSTRACT

Progressive Surgical Distraction Device (PSDD) intended for enabling gradual atraumatic access to cavities and zones inside the human body and for allowing controlled extraction of the liquids contained therein, particularly open foetal surgery and neurosurgery. It is composed of a set of cylindrical and semi-cylindrical parts of increasing diameters and decreasing lengths that fit into each other telescopically, each enabling the successive extraction of the inner part. The progressive distraction of the muscular fibres does not subject them to traction, avoids haemorrhages and allows the controlled diversion of liquids without accumulation of residues. The last part of the sequence is designed to allow an optimum mobilisation of the clamping system via directly viewed control of the intervention zone and surgical field.

### SECTOR AND OBJECT OF THE INVENTION

The object of the invention falls within the surgical device sector. Specifically, it is a surgical distraction device consisting of a set of telescopic tubes wherein each of the constituent parts of the set have correlative inner and outer diameters, conferring a progressive character and allowing atraumatic access to the surgical zone. The surgical distraction device object of the present invention is applicable in diverse operations, particularly in open foetal surgery.

### STATE OF THE ART

In recent years, open foetal surgery has become increasingly important not only as a procedure that saves lives, but also for providing the possibility of correcting foetal defects, interventions which were considered high-risk and even ethically questionable. The turning point came with the introduction of the correction of foetal myelomeningocele [Tulipan N. Et al. “Intrauterine closure of myelomeningocele: an update”; Neurosurg Focus 16 (2): Article 2, 2004]. Intrauterine repair of non-lethal malformations by open access through a hysterotomy has become a therapeutic option and its increasing use has offered opportunities to improve surgical techniques and the corresponding instrumentation, converting these types of interventions into an increasingly less exceptional practice.

Damaged and torn uterine fibres are one of the main problems that must be solved during open foetal surgery. To this end, the ideal uterine access technique must be fast, simple, atraumatic and bloodless. Uterine entry is carried out through a Seldinger-type access. The uterus can then be opened using different techniques; those that use some type of dilator do so by introducing a single, thick trocar [Tulipan N. et al. “The Tulipan-Burner Trocar for uterine entry during foetal surgery”; Am. J. Obstet. Gynecol. 181; 1188-1191 (1999) and Almodin C. G. et al. “The Almodin-Moron Trocar for Uterine Entry during Foetal Surgery”; Foetal Diagn. Ther. 2006; 21; 414-417].

The surgical distraction device object of the present invention has the following advantages over known devices:
it is a progressive dilation system ranging from the minimum calibre required for controlled drainage which allows diversion of amniotic fluid to the maximum calibre, where the calibre of the cavity produced allows optimum mobilisation of the clamping system and control via directly viewed control of foetal parts;
the progressive distraction of muscular fibres does not subject them to the traction of state-of-the-art devices, particularly that of Brazilian patent P10402885 (Almodin trocar), thereby preventing haemorrhage of the uterine wall while offering an optimum operating zone;
the progressive system of variable length from the interior to the exterior enables manual control of the distraction, as there cannot be distal support (the foetal parts should not be reached), due to which it is necessary to maintain the systems in place during the dilation process;
the diversion of amniotic fluid is performed in a direct and closed manner during the surgical process;
the configuration of the device prevents accumulation of residues therein.

There are other types of devices, such as that of U.S. Pat. No. 6,623,502, relative to a guidance system for laparoscopic surgery. The device does not allow the Seldinger technique and would therefore not be usable in the types of interventions wherein the progressive distraction system object of the present invention can be used, particularly in intrauterine foetal surgery.

### EXPLANATION OF THE INVENTION

The object of the present invention is a progressive surgical distraction device for atraumatic access constituted by a set of parts in the form of cylindrical and semi-cylindrical hollow tubes with rounded edges and without rims or flanges that define an ordered sequence of increasing diameters. Said set comprises between three and fourteen tubes, of which all except that with the greatest outer diameter have a truncated cone-shaped end which is telescopic, each of the parts of the set having correlative inner and outer diameters, in such a manner that each part embraces the previous part and is embraced by the next part.

Each of the parts of the set has a total length greater than that of the part that embraces it, allowing extraction of those parts with a smaller diameter by means of traction of the rear part thereof, which projects from the rear of the next part of larger diameter introduced. Those parts of the set with a truncated cone-shaped end have the following characteristics:
smaller inner diameters comprised between 1.4 mm and 40 mm, preferably between 1.4 mm and 16 mm;
lengths comprised between 500 mm and 120 mm, preferably between 300 mm and 120 mm;
the cylindrical end of the parts are equipped with at least one pass-through hole having a diameter comprised between 0.5 mm and 1 mm, which allows fixation, fastening or extraction thereof using any other surgical device, particularly silk or wire; and
optionally, the parts have a thread at the cylindrical end to allow closure thereof by means of plugs or connection to other devices.

The part with the largest diameter does not have a truncated cone-shaped end and is formed of two parts that are welded together:
a) a hollow semi-cylinder having an inner diameter larger than any of the parts with a truncated cone-shaped end and length comprised between 50 mm and 125 mm;
b) a semi-washer welded externally to the semi-cylinder at the rear end thereof with a height of between the eighth and and the tenth of the outer diameter of the hollow semi-cylinder.
   All the parts of the set have wall thickness comprised between 0.3 mm. and 2 mm., being said parts made in medical-grade stainless steel.

In a first preferred embodiment of the invention, the assembly is composed of the following groups of parts:
a) a first cylindrical part having a truncated cone-shaped end with the following characteristics:
   a minor inner diameter comprised between 2 mm and 3 mm;
   a minor outer diameter comprised between 4 mm and 5 mm;
   a major inner diameter, identical to that of the cylindrical portion, of 6 mm;
   a major outer diameter, identical to that of the cylindrical portion, of 7.8 mm;
   a truncated cone-shaped portion between 30 mm and 50 mm in length;
   a total length of the part of 280 mm;
   an angle between the truncated cone-shaped and cylindrical portions comprised between 5° and 8°.
b) a group of four cylindrical parts having a truncated cone-shaped end with the following characteristics:
   inner diameters comprised between 7.8 mm and 16 mm;
   outer diameters comprised between 9.8 mm and 20 mm;
   total lengths comprised between 240 mm and 120 mm;
   truncated cone-shaped portion between 4 mm and 5 mm in length;
   truncated cone-shaped portion terminations in the form of an inner cone having angles with respect to the cross-section comprised between 40° and 60° and heights comprised between 0.7 mm and 0.8 mm;
   an orifice having a diameter of 1 mm at the cylindrical end of each of the parts;
   wall thicknesses comprised between 0.4 mm and 2 mm.
c) a semi-cylindrical part having an inner diameter of 22.5 mm, an outer diameter of 22.5 mm, a wall thickness of 1.5 mm and a length comprised between 50 mm and 125 mm having, at one of its ends, a semi-washer welded externally to the semi-cylindrical part, said semi-washer having an inner diameter of 22.5 mm, an outer diameter of 40 mm and a height of 3 mm.

In a second embodiment of the invention, the assembly is composed of the following groups of parts:
a) a group of six cylindrical parts with a truncated cone-shaped end having:
   inner diameters comprised between 1.4 mm and 6 mm;
   outer diameters comprised between 2 mm and 7.8 mm;
   total lengths comprised between 300 mm and 250 mm;
   truncated cone-shaped portions between 1 mm and 6 mm in length;
   wall thicknesses comprised between 0.3 mm and 0.9 mm;
   an orifice having a diameter comprised between 0.5 mm and 1 mm at the cylindrical end of each of the parts.
b) a group of four cylindrical parts having a truncated cone-shaped end with the following characteristics:
   inner diameters comprised between 7.8 mm and 16 mm;
   outer diameters comprised between 9.8 mm and 20 mm;
   total lengths comprised between 240 mm and 120 mm;
   truncated cone-shaped portions between 4 mm and 5 mm in length;
   truncated cone-shaped portion terminations in the form of an inner cone having angles with respect to the cross-section comprised between 40° and 60° and heights comprised between 0.7 mm and 0.8 mm;
   an orifice 1 mm in diameter at the cylindrical end of each of the parts;
   wall thicknesses comprised between 0.4 mm and 2 mm.
c) a semi-cylindrical part having an inner diameter of 22.5 mm, an outer diameter of 25.5 mm, a wall thickness of 1.5 mm and a length comprised between 50 mm and 125 mm having, at one of its ends, a semi-washer welded externally to the semi-cylindrical part, said semi-washer having an inner diameter of 25.5 mm, an outer diameter of 40 mm and a height of 3 mm.

Another object of the present invention consists of the use of the distraction device for accessing zones inside the lungs and digestive system and specifically the use of the distraction device according to the two preferred embodiments in intrauterine foetal surgery and for accessing cavities inside the head and heart.

### BRIEF DESCRIPTION OF THE FIGURES

FIG. 1 shows an overall view of the progressive surgical distraction device with all of its elements.

FIG. 2 shows an overall view of element 1 of the distraction device consisting, in this configuration, of six parts (1-C-1 to 1-C-6) of increasing diameter.

FIGS. 3: A) shows element 1 of the distraction device consisting of a single 280 mm part (part 1-A) having a 40 mm truncated cone-shaped portion; B) shows a detailed view of the end of the cylindrical portion of part 1-A, which has a pass-though hole; C) and D) show detailed elevational and plan views of the truncated cone-shaped portion of part 1-A as element 1 of the distraction device.

FIGS. 4: A) shows element 1 of the distraction device consisting of a single 280 mm part (part 1-B) having a 30 mm truncated cone-shaped portion; B) shows a detailed view of the end of the cylindrical portion of part 1-B, which has a pass-through orifice; C) and D) show detailed elevational and plan views of the truncated cone-shaped portion of part 1-A as element 1 of the distraction device.

FIGS. 5: A) and B) show views of part 1-C-1 of element 1 of the distraction device; C) shows a detailed view off the end of the cylindrical portion of part 1-C-1 of element 1, which has a pass-through orifice. FIGS. 6: A) and B) show views of part 1-C-2 of element 1 of the distraction device; C) shows a detailed view of the end of the cylindrical portion of part 1-C-2 of element 1, which has a pass-through orifice.

FIGS. 7: A) and B) show views of part 1-C-3 of element 1 of the distraction device; C) shows a detailed view of the end of the cylindrical portion of part 1-C-3 of element 1, which has a pass-through orifice.

FIGS. 8: A) and B) show views of part 1-C-4 of element 1 of the distraction device; C) shows a detailed view of the end of the cylindrical portion of part 1-C-4 of element 1, which has a pass-through orifice.

FIGS. 9: A) and B) show views of part 1-C-5 of element 1 of the distraction device; C) shows a detailed view of the end of the cylindrical portion of part 1-C-5 of element 1, which has a pass-through orifice.

FIGS. 10: A) and B) show views of part 1-C-6 of element 1 of the distraction device; C) shows a detailed view of the end of the cylindrical portion of part 1-C-6 of element 1, which has a pass-through orifice.

FIGS. 11: A) and B) show views of element 2 of the distraction device; C) shows a detailed view of the truncated cone-shaped portion of element 2 of the distraction device.

FIGS. 12: A) and B) show views of element 3 of the distraction device; C) shows a detailed view of the truncated cone-shaped portion of element 3 of the distraction device.

FIGS. 13: A) and B) show views of element 4 of the distraction device; C) shows a detailed view of the truncated cone-shaped portion of element 4 of the distraction device.

FIGS. 14: A) and B) show views of element 5 of the distraction device; C) shows a detailed view of the truncated cone-shaped portion of element 5 of the distraction device.

FIGS. 15: A), B) and C) show plan, elevational and profile views of one of the configurations of element 6 (part 6-A) of the distraction device.

FIGS. 16: A A), B) and C) show plan, elevational and profile views of another of the configurations of element 6 (part 6-B) of the distraction device.

### DETAILED DESCRIPTION OF THE INVENTION

The progressive surgical distraction device object of the present invention is composed of a set of elements in the form of cylindrical and semi-cylindrical hollow tubes which define an ordered sequence. These elements have a truncated cone-shaped end or front part (“point”) and a cylindrical (from part 1-A to part 5) or semi-cylindrical straight body (parts 6-A and 6-B), the rear part of which (of all the parts) ends in a straight section perpendicular to its longitudinal axis. The last part (parts 6-A or 6-B) is a semi-cylinder which allows support and sliding thereon of a “cutting and clamping gun” type surgical instrument or similar. This last part has a semi-circular rim around the rear part thereof to rest on the outer surface of the patient's body, allow fixation thereof to other surgical field supports and prevent sliding towards the interior of the body.

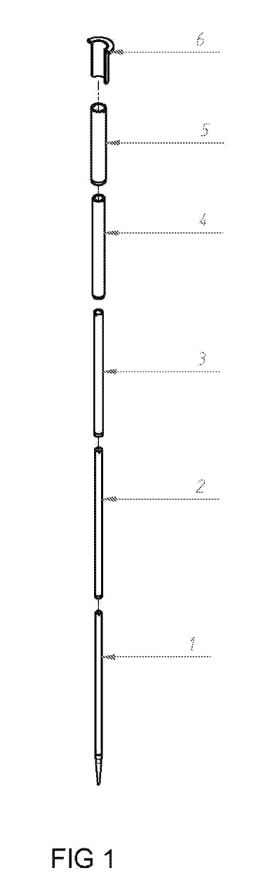

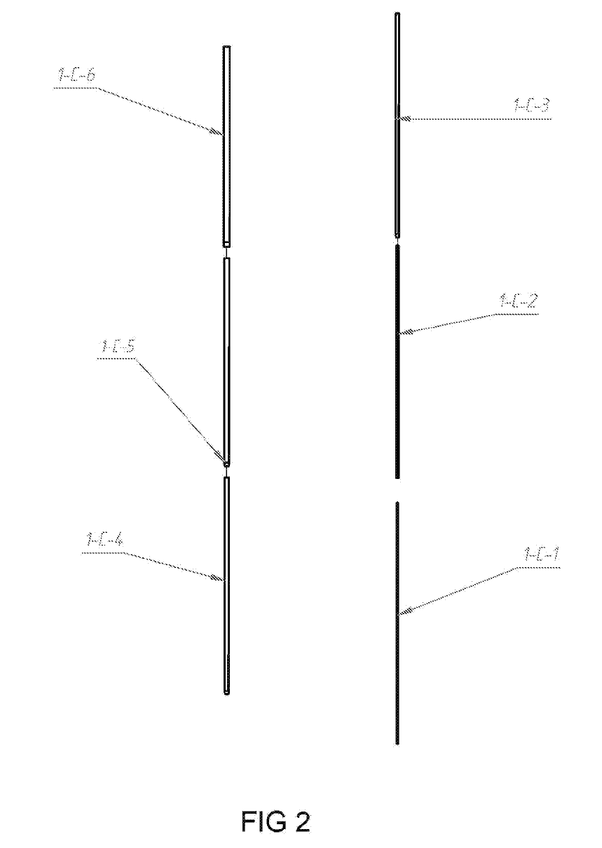

All the parts are manufactured from medical-grade stainless steel (316L steel or similar) and, with the organisation and assembly represented in the figures (particularly FIGS. 1 and 2), define a system having the following characteristics:
a) Ordered modular: the set of parts forms an ordered sequence to be used starting from that having the smallest diameter (parts 1A, 1B or 10) and, subsequently, in ascending order of diameters (as detailed in the embodiment of the invention) to that with the largest diameter (parts 6A or 6B).
b) Telescopic: the parts have correlative inner and exterior diameters, in such a manner that each part is housed by the previous part and houses the next part, sliding tightly into each other (due to the polishing of the inner and outer surfaces thereof), allowing sequential introduction as explained in detail in the next point, similar to that known as the “Seldinger method” for introduction catheters. Exceptionally, parts 6-A or 6-B have an inner diameter slightly larger than that of part 5, which it houses. This is justified by the need for part 6, on being the last in the use sequence and having a semi-cylindrical shape, to be able to embrace and allow extraction of part 5 smoothly and without exerting any pressure whatsoever.
c) Progessive and Gradual: the parts are introduced into the body following the indicated sequence of increasing diameters indicated in the previous point. In this manner, the initial orifice opened in the tissue is that corresponding to the smallest diameter and, which is progressively enlarged by means of the insertion and progressive withdrawal of the parts with a larger diameter until reaching that of the part with the largest diameter, thereby achieving atraumatic access to the desired destination point. The aperture of the orifice is progressive because each part has an inner diameter larger than that of the previous part and gradual because the entry points of each of the parts have a truncated cone shape, so that tissue dissection is smooth and atraumatic.
d) Retractable: each part has a total length greater than the next part, in such a manner that the (previous) part can be extracted, as in the aforementioned “Seldinger method”, by means of traction of the rear part thereof that projects from the rear part of the next part introduced.
e) Equipped with a safety fixation: all the parts have one or two holes at their rear end 0.5-1.0 mm in diameter which allow fixation of the part by means of silk or wire or any other surgical device and which allow fixation, securing or removal (extraction) thereof.
f) Connectable to threaded elements: each part has a thread (optional) on the rear end to allow connection thereof to tubes, valves, plugs and any other similar device. This enables secure extraction of the liquid contained within the zone (cavity, tissue, etc.) which is accessed through the front end (point) of the corresponding part.
g) Easily cleaned: the interior of the parts is a smooth cylinder having truncated cone-shaped points without bends, edges or rims where deposits of any kind can be accumulated, cleanable and sterilisable using regular surgical instrument cleaning processes.
h) Adaptable to the specific anatomic and physiological characteristics of each case by:
   h.1) Adjusting the sharpness of the first part inserted (Part 1): there are two parts (1-A and 1-B) which have undergone different machining processes that differ in the sharpness of the point, which is defined by the length of the corresponding truncated cone, having the same total length and diameters as the front part (point) and cylinder (body and rear part). Part 1-A has a possible truncated cone length of 40-60 mm (the part is represented with a length of 40 mm) and is, therefore, sharper. Part 1-B has a possible cone length of 10-30 mm (the part is represented with a length of 30 mm), therefore being less sharp.
   h.2) Adjusting the extraction flow rate of the liquid contained in the cavity or zone wherein the point of the part is inserted: there is a set of parts (parts 1-C-1 to 1-C-6) the point inner diameter of which ranges from 1.4 mm to 7.8 mm. Therefore, the flow rate can be gradually adjusted through the use of several diameters, enabling control of the pressure of the liquid inside the cavity or enclosure wherefrom it is extracted.
   h.3) Adjusting the thickness of the tissues (muscles, fat, etc.) penetrated: the last part (parts 6-A and 6-B) is available in different lengths ranging from 50-125 mm, so that it can adapt to the thickness of the tissue from the exterior of the patient to the destination zone. This will enable atraumatic access to the desired cavity without pushing the edges or layers of the tissues or elements penetrated inwards and without exerting pressure or compressing elements, organs or tissues in the interior of the cavity accessed.
   h.4) Scalability: in order to increase its range of applicability to other specific fields (thoracic surgery, heart surgery, . . . ) all the parts can be manufactured in other lengths (ranging from 280-500 mm) and larger and smaller diameters (1.4-60 mm), maintaining the proportionality therebetween.

### EMBODIMENT OF THE INVENTION

All of the parts are manufactured from medical grade stainless steel (316L steel or similar). Parts 1-C1, 1-C-2, 1-C-3, 1-C-4, 1-C-5, 1-C-6 and elements 2, 3, 4 and 5 are manufactured using, in each case, a rod having a rounded entering edge, generating an outer truncated cone and subsequently dulling the entering edge to make it blunt. All sharp edges are rounded.

Parts 1-A and 1-B will be manufactured joining the two different parts. On one hand, a hollow steel rod. On the other, a truncated cone, the inner and outer diameters of the base of which will be the same as those of the aforementioned rod. These two rods will be welded together forming a single element.

Parts 6-A and 6-B will also be manufactured welding two different parts: a hollow semi-cylinder and a part that will act as an outer edge, also being a semi-cylinder but much lower in height than its inner and outer diameters. All the parts will be externally polished in such a manner that they slide into each other in an optimum manner.

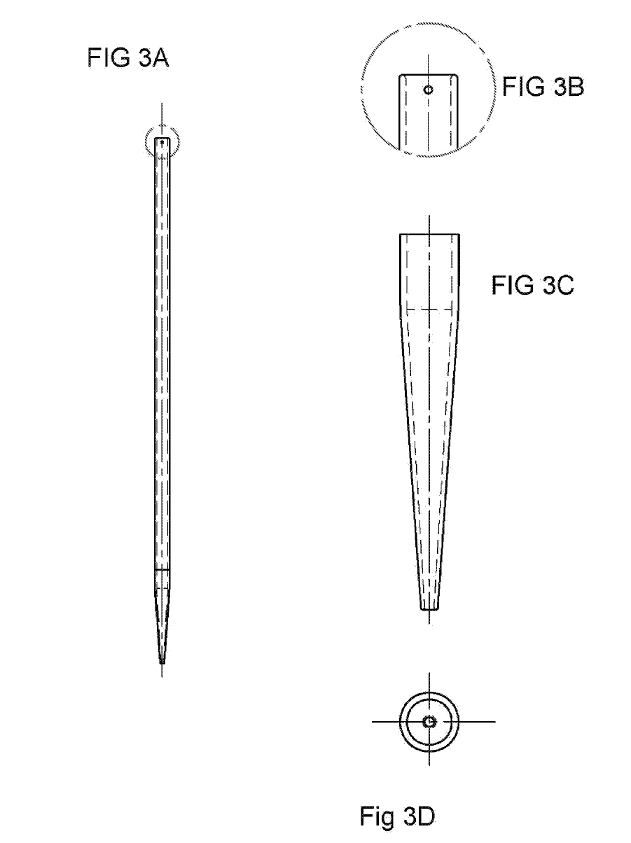

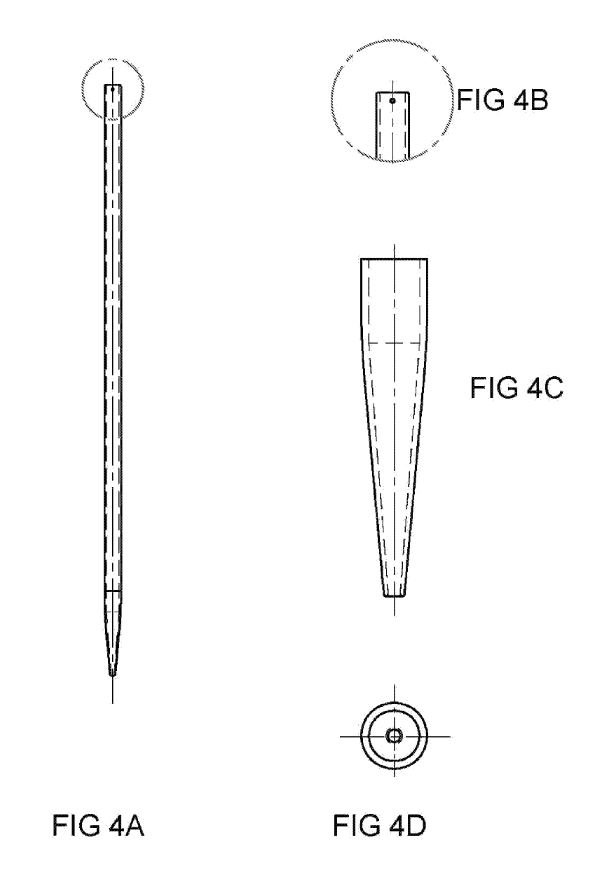

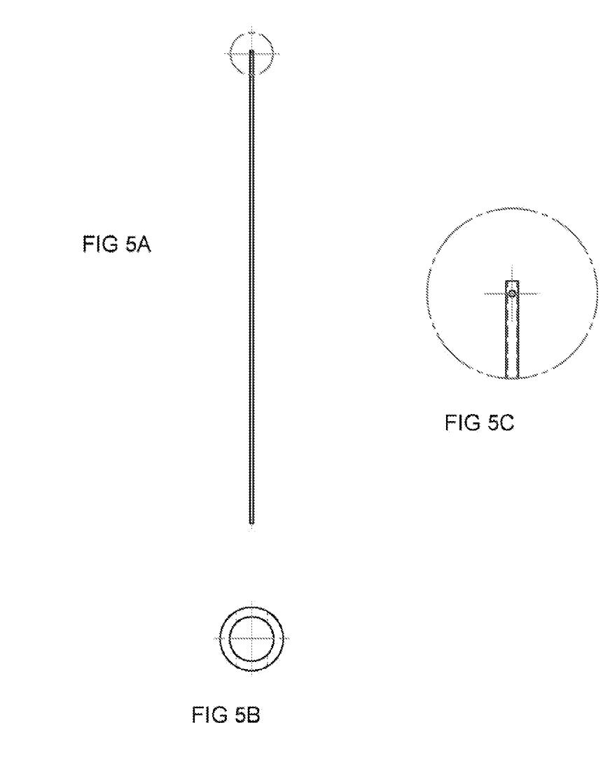

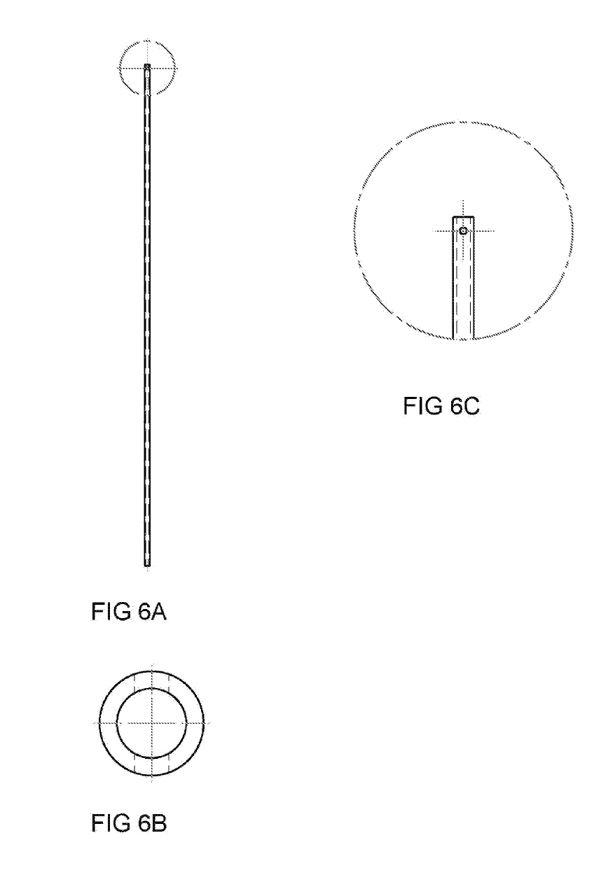

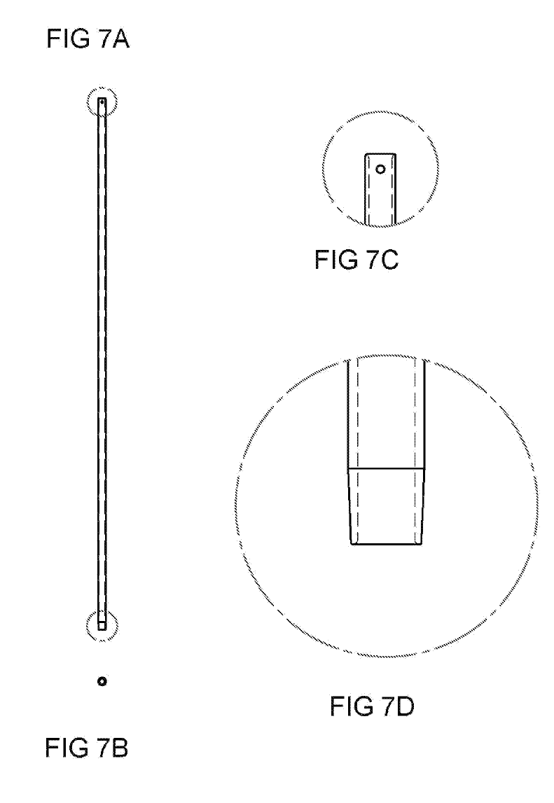

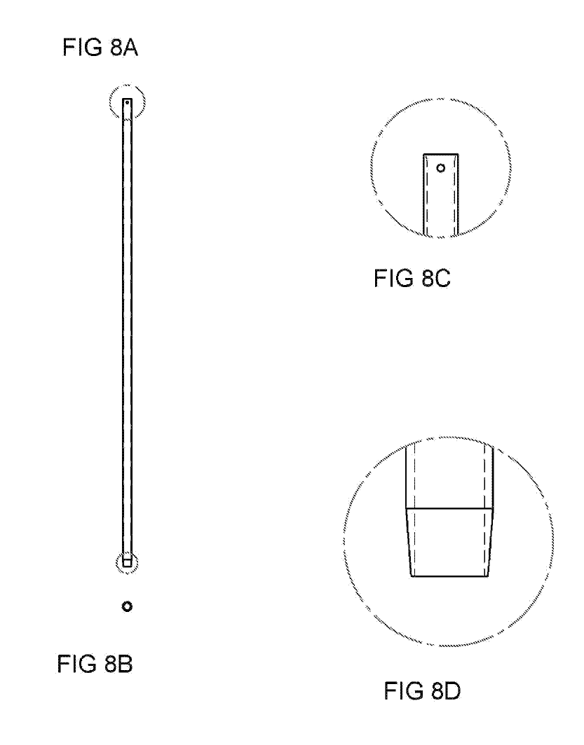

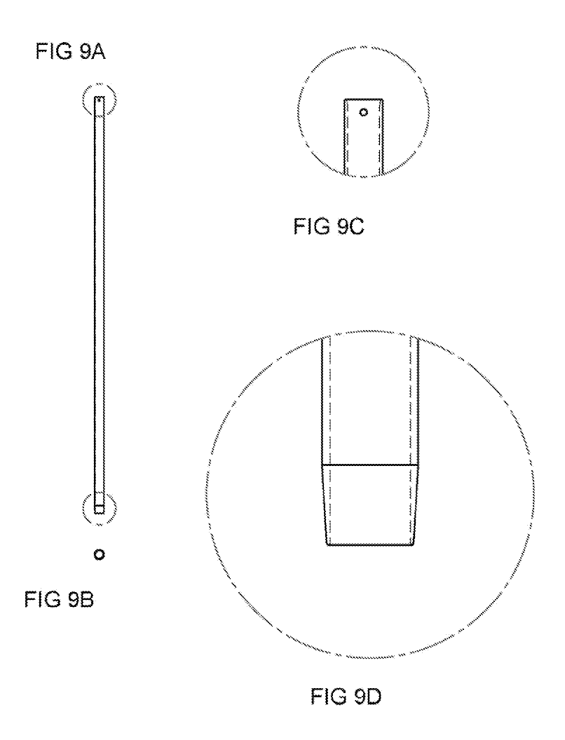

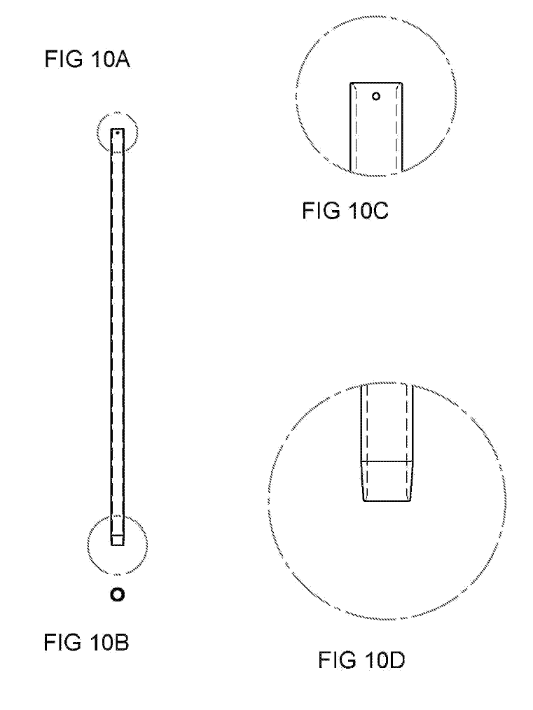

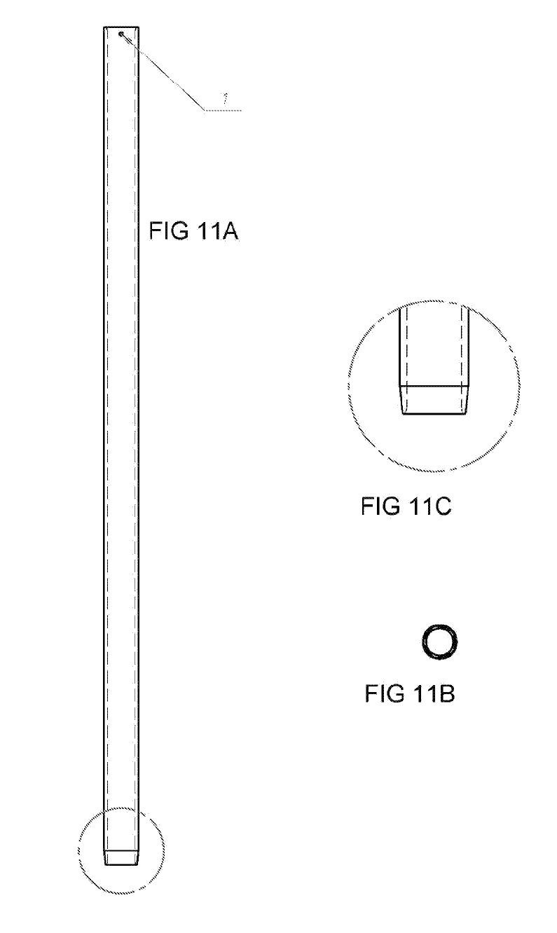

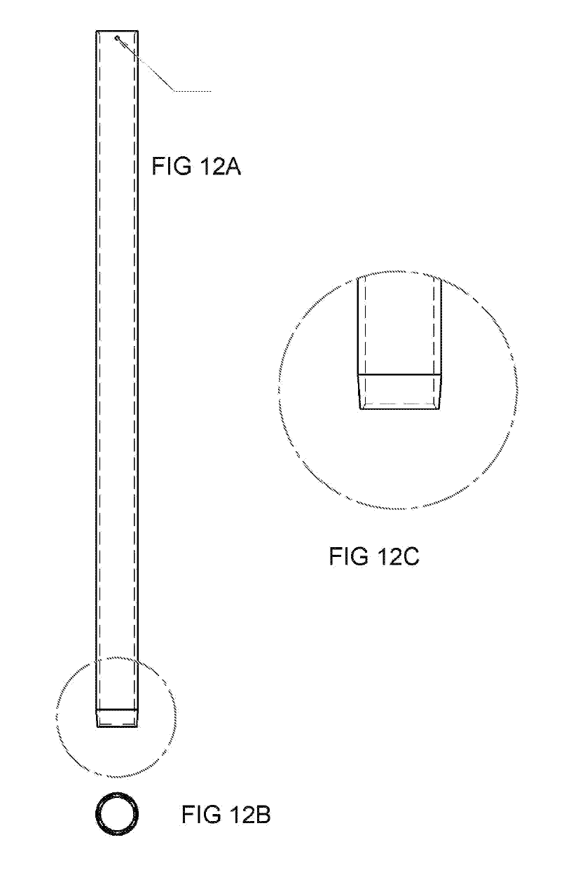

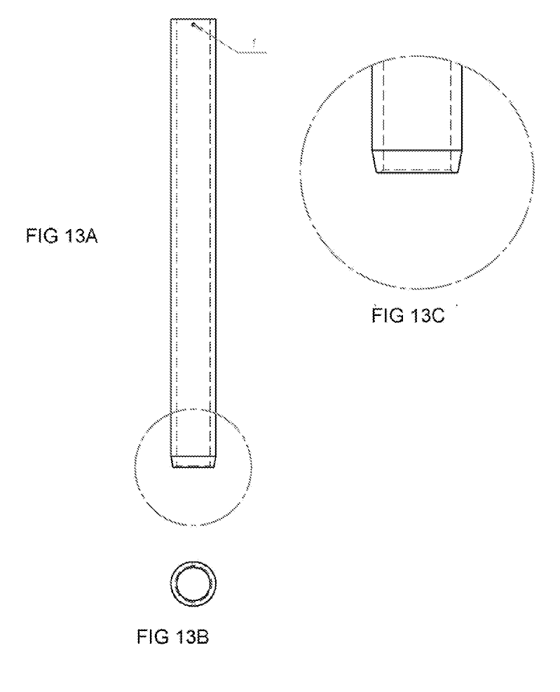

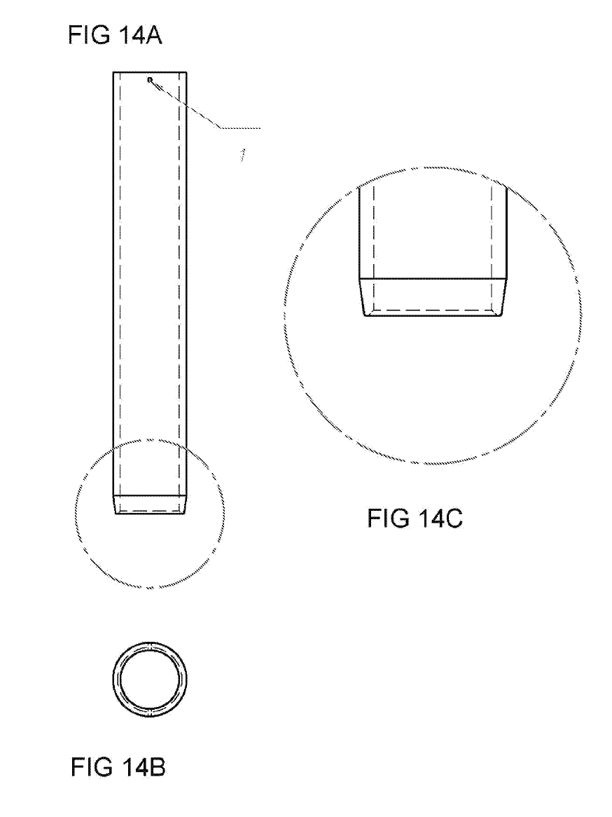

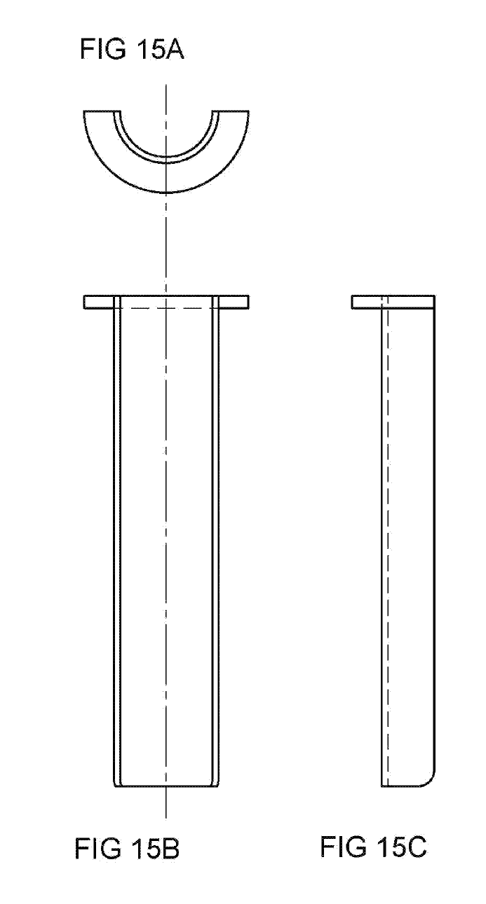

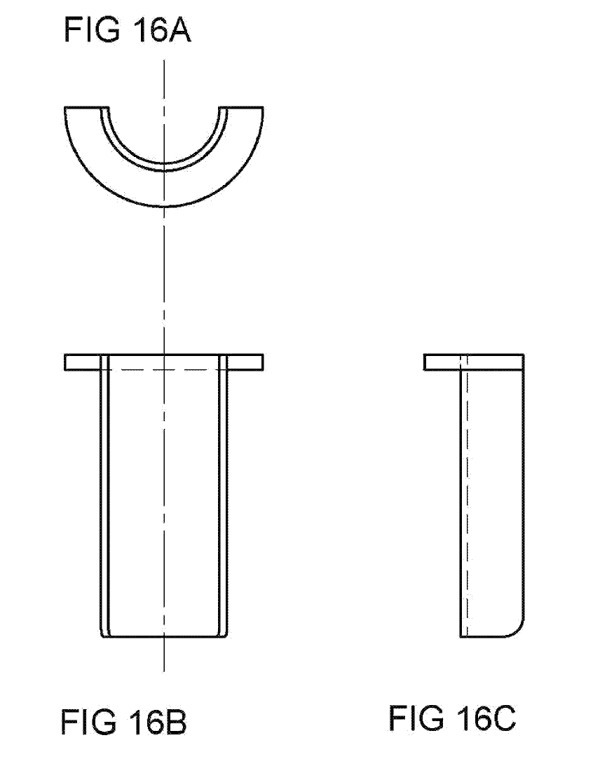

The main characteristics of each of the parts (represented in the attached figures) are explained in detail below:
Part 1-A (FIG. 3) is composed of two parts. The first is a hollow truncated cone, having a constant wall thickness, the minor outer diameter of which is 4.4 mm and the major outer diameter 7.8 mm. It has total length corresponding to the sum of the length of the truncated cone plus a certain cylinder length (to enable machining and welding thereof) of 5-15 mm. In the represented part these dimensions are 40 mm+10 mm (40 mm are truncated cone-shaped and 10 mm are cylindrical), producing an angle of 5° measured at the theoretical cone vortex. The second part is a hollow cylinder having a wall thickness equal to that of the truncated cone and a total length of 230 mm. This results in a total part length of 280 mm. The two parts of the part are welded together and the joint between them polished to the same grade as the rest of the part. The entering edge of the part, i.e. the minor diameter of the truncated cone, has been dulled to make it blunt.
   Part 1-B (FIG. 4) is composed of two parts. The first is a hollow truncated cone, having a constant wall thickness, the minor outer diameter of which is 4.8 mm and the major outer diameter 7.8 mm. It has a total length corresponding to the sum of the length of the truncated cone plus a certain length of cylinder (to enable machining and welding thereof) of 5-15 mm. In the represented body these dimensions are 30 mm+10 mm (30 mm are truncated cone-shaped and 10 mm cylindrical), producing an angle of 7.4° measured at the theoretical cone vortex. The second part is a hollow cylinder, having a wall thickness equal to that of the truncated cone and a total length of 240 mm. This results in a total part length of 280 mm. The two parts of the part are welded together and the joint therebetween polished to the same grade as the rest of the part. The entering edge of the part, i.e. the minor diameter of the truncated cone, has been dulled to make it blunt. The main difference with part 1-A is that its conicity angle is greater.
The set of parts from 1-C.1 to 1-C-6 perform a function equivalent to that of parts 1-A and 1-B. They are a subset of rods formed of hollow cylinders having a constant thickness and increasing diameter, the final diameter of which (7.8 mm) coincides with the outer diameter of parts 1-A and 1-B.
Part 1-C.1 (FIG. 5) has an outer diameter of 2 mm, a thickness of 0.3 mm and a total length of 300 mm. A truncated cone has been carved on its front end, the minor diameter of which is 1.4 mm, with a total length of 1 mm. An orifice has been made on its rear end with a diameter of 0.5 mm for fixation thereof.
Part 1-C-2 (FIG. 6) has an outer diameter of 3 mm, a thickness of 0.5 mm and a total length of 290 mm. A truncated cone has been carved on its front end, the minor diameter of which is 2 mm, with a total length of 1 mm. An orifice has been made on its rear end with a diameter of 1 mm for fixation thereof.
Part 1-C.3 (FIG. 7) has a diameter of 4 mm, a thickness of 0.5 mm and a total length of 280 mm. A truncated cone has been carved on its front end, the minor outer diameter of which is 3.7 mm (inner diameter of 3 mm), with a total length of 4 mm. An orifice has been made on its rear end with a diameter of 1 mm for fixation thereof.
Part 1-C-4 (FIG. 8) has a diameter of 5 mm, a thickness of 0.5 mm and a total length of 270 mm. A truncated cone has been carved on its front end, the minor outer diameter of which is 4.4 mm (inner diameter of 4 mm), with a total length of 4 mm. An orifice has been made on its rear end with a diameter of 1 mm for fixation thereof.
Part 1-C.5 (FIG. 9) has a diameter of 6 mm, a thickness of 0.5 mm and a total length of 260 mm. A truncated cone has been carved on its front end, the minor outer diameter of which is 5.4 mm (inner diameter of 5 mm), with a total length of 5 mm. An orifice has been made on its rear end with a diameter of 1 mm for fixation thereof.
Part 1-C-6 (FIG. 10) has a diameter of 7.8 mm, a thickness of 0.9 mm and a total length of 250 mm. A truncated cone has been carved on its front end, the minor outer diameter of which is 7 mm (inner diameter of 6 mm), with a total length of 6 mm. An orifice has been made on its rear end with a diameter of 1 mm for fixation thereof.
The following elements, from 2 to 5, also continue from this sequence and can be used subsequently to the subset of parts 1-C or after parts 1-A or 1-B.
Element 2 (FIG. 11) has a diameter of 9.8 mm, a thickness of 0.4 mm and a total length of 240 mm. A truncated cone has been carved on its front end, the minor outer diameter of which is 9 mm (inner diameter of 7.8 mm), with a total length of 4 mm. An orifice has been made on its rear end with a diameter of 1 mm for fixation thereof.
Element 3 (FIG. 12) has a diameter of 12 mm, a thickness of 1.1 mm and a total length of 200 mm. A truncated cone has been carved on its front end, the minor outer diameter of which is 11.3 mm (inner diameter of 9.8 mm), with a total length of 5 mm. An orifice has been made on its rear end with a diameter of 1 mm for fixation thereof. The mouth ends in an inner cone having an angle of 60° with respect to the longitudinal axis and a height of 0.8 mm with respect to the cross-section.
Element 4 (FIG. 13) has a diameter of 16 mm, a thickness of 2 mm and a total length of 160 mm. A truncated cone has been carved on its front end, the minor outer diameter of which is 14.5 mm (inner diameter of 12 mm), with a total length of 4 mm. An orifice has been made on its rear end with a diameter of 1 mm for fixation thereof. The mouth ends in an inner cone having an angle of 60° with respect to the longitudinal axis and a height of 0.5 mm with respect to the cross-section.
Element 5 (FIG. 14) has a diameter of 20 mm, a thickness of 2 mm and a total length of 120 mm. A truncated cone has been carved on its front end, the minor outer diameter of which is 18.6 mm (inner diameter of 16 mm), with a total length of 4 mm. An orifice has been made on its rear end with a diameter of 1 mm for fixation thereof. The mouth ends in an inner cone having an angle of 45° and a height of 0.75 mm with respect to the cross-section.
Parts 6-A and 6-B are similar therebetween; only the total length of the part varies for use in different circumstances in accordance with the thickness of the tissues to be penetrated.
Part 6-A (FIG. 15) is formed of two parts which are welded together. The first is a hollow semi-cylinder obtained from cutting and polishing a steel rod, having an outer diameter of 25.5 mm, a thickness of 1.5 mm and a length of 120 mm. The second part is a semi-washer, i.e. a hollow semi-cylinder the height of which is much smaller than its inner or outer diameters. Specifically, its height will be 3 mm, its outer diameter 40 mm and its inner diameter 25.5 mm, identical to the outer diameter of the semi-cylinder. The semi-washer will be welded externally to the semi-cylinder on its rear end. The front end of the semi-cylinder will have rounded edges. The corners that form its straight sides with the semi-circular sides will be rounded with a curvature radius of 4 mm.
Part 6-B (FIG. 16) is formed of two parts which are welded together. The first is a hollow semi-cylinder obtained from cutting and polishing a steel rod, having an outer diameter of 25.5 mm, a thickness of 1.5 mm and a length of 57 mm. The second part is a semi-washer, i.e. a hollow semi-cylinder the height of which is much smaller than its inner or outer diameters. Specifically, its height will be 3 mm, its outer diameter 40 mm and its inner diameter 25.5 mm, identical to the outer diameter of the semi-cylinder. The semi-washer will be welded externally to the semi-cylinder on its rear end. The front end of the semi-cylinder will have rounded edges. The corners that form its straight sides with the semi-circular sides will be rounded with a curvature radius of 4 mm. The only difference with part 6-A is the length of the semi-cylinder.

1. A progressive surgical distraction device for atraumatic access comprising of a set of parts in the form of cylindrical and semi-cylindrical hollow tubes having rounded edges without rims or flanges that define an ordered sequence with increasing diameters, characterised in that
a) said set of tubes comprises between three and fourteen tubes, of which all except that with the largest outer diameter have a truncated cone-shaped end and
b) the set is telescopic, each of the constituent parts of the set having correlative inner and outer diameters, in such a manner that each houses the previous part and is housed inside the next part.

2. A progressive surgical distraction device for atraumatic access, according to claim 1, wherein each of the parts of the set have a total length greater than the part that houses it, allowing extraction of the parts with smaller diameter by traction of the rear part thereof, which projects from the rear part of the next part of larger diameter introduced.

3. A progressive surgical distraction device for atraumatic access, according to claim 1, wherein the minor inner diameters of the parts of the set having a truncated cone-shaped end are between 1.4 mm and 40 mm.

4. A progressive surgical distraction device for atraumatic access, according to claim 3, wherein the minor inner diameters of the parts of the set having a truncated cone-shaped end are between 1.4 mm and 16 mm.

5. A progressive surgical distraction device for atraumatic access, according to claim 1, wherein the lengths of the parts of the set having a truncated cone-shaped are between 500 mm and 120 mm.

6. A progressive surgical distraction device for atraumatic access, according to claim 5, wherein the lengths of the parts of the set having a truncated cone-shaped end are between 300 mm and 120 mm.

7. A progressive surgical distraction device for atraumatic access, according to claim 1, wherein the parts of the set having a truncated cone-shaped end have at least one orifice on the cylindrical end between 0.5 mm and 1 mm allowing fixation, fastening or extraction thereof using any other surgical device, particularly silk or wire.

8. A progressive surgical distraction device for atraumatic access, according to claim 1, wherein the parts of the set having a truncated cone-shaped end have a thread on the cylindrical end which allows closure thereof by means of plugs or connection thereof to other devices.

9. A progressive surgical distraction device for atraumatic access, according to claim 1, wherein the part with the largest inner diameter is formed of two parts which are welded together:
a) a hollow semi-cylinder having an inner diameter larger than that of any of the parts with a truncated cone-shaped end and length between 50 mm and 125 mm;
b) a semi-washer welded externally to the semi-cylinder on its rear end having a height between the eighth and the tenth of the outer diameter of the hollow semi-cylinder.

10. A progressive surgical distraction device for atraumatic access, according to claim 1, wherein the wall thicknesses of all the constituent parts of the set are between 0.3 mm and 2 mm.

11. A progressive surgical distraction device for atraumatic access, according to claim 1, wherein the parts are manufactured from medical grade stainless steel.

12. A progressive surgical distraction device for atraumatic access, according to claim 1, wherein the set consists of the following groups of parts:
a) a first cylindrical part having a truncated cone-shaped end with the following characteristics:
a minor inner diameter between 2 mm and 3 mm;
a minor outer diameter between 4 mm and 5 mm;
a major inner diameter equal to that of the cylindrical portion of 6 mm;
a major outer diameter equal to that of the cylindrical portion of 7.8 mm;
a truncated cone-shaped portion length comprised between 30 mm and 50 mm;
a total part length of 280 mm;
an angle between the truncated cone-shaped and cylindrical portions between 5° and 8°.
b) a group of four cylindrical parts having a truncated cone-shaped end with the following characteristics:
inner diameters between 7.8 mm and 16 mm;
outer diameters between 9.8 mm and 20 mm;
total lengths comprised between 240 mm and 120 mm;
truncated cone-shaped portion lengths between 4 mm and 5 mm;
truncated cone-shaped terminations in the form of an inner cone having angles with respect to the cross-section between 40° and 60° and heights between 0.7 mm and 0,8 mm.;
a 1 mm diameter orifice on the cylindrical end of each of the parts; and
wall thicknesses between 0.4 mm and 2 mm.
c) a semi-cylindrical part having an inner diameter of 22.5 mm, an outer diameter of 25.5 mm, a wall thickness of 1.5 mm and a length between 50 mm and 125 mm having, on one of its ends, a semi-washer welded externally to the semi-cylindrical part, said semi-washer having an inner diameter of 25.5 mm, an outer diameter of 40 mm and a height of 3 mm.

13. A progressive surgical distraction device for atraumatic access, according to claim 1, wherein the set is composed of the following groups of parts:
a) a group of six cylindrical parts having a truncated cone-shaped end with:
inner diameters between 1.4 mm and 6 mm;
outer diameters between 2 mm and 7.8 mm;
total lengths between 300 mm and 250 mm;
truncated cone-shaped portion lengths between 1 mm and 6 mm;
wall thicknesses between 0.3 mm and 0.9 mm; and
an orifice having a diameter between 0.5 mm and 1 mm on the cylindrical end of each of the parts.
b) a group of four cylindrical parts having a truncated cone-shaped end with the following characteristics:
inner diameters between 7.8 mm and 16 mm;
outer diameters between 9.8 mm and 20 mm;
total lengths between 240 mm and 120 mm;
truncated cone-shaped portion lengths between 4 mm and 5 mm;
truncated cone-shaped terminations in the form of an inner cone having angles with respect to the cross-section between 40° and 60° and heights between 0.7 mm and 0.8 mm;
an orifice having a diameter of 1 mm on the cylindrical end of each of the parts;
wall thicknesses between 0.4 mm and 2 mm.
c) a semi-cylindrical part having an inner diameter of 22.5 mm, an outer diameter of 25.5 mm, a wall thickness of 1.5 mm and a length between 50 mm and 125 mm having, on one of its ends, a semi-washer welded externally to the semi-cylindrical part, said semi-washer having an inner diameter of 25.5 mm, an outer diameter of 40 mm and a height of 3 mm.

14. Use of a surgical distraction device, according to claim 1, for accessing zones inside the lungs and digestive system.

15. Use of a progressive surgical distraction device, according to claim 12, in intrauterine foetal surgery.

16. Use of a progressive surgical distraction device, according to claim 12, for accessing cavities inside the head.

17. Use of a progressive surgical distraction device, according to claim 12, for accessing cavities inside the heart.

18. Use of a progressive surgical distraction device, according to claim 13, in intrauterine foetal surgery.

19. Use of a progressive surgical distraction device, according to claim 13, for accessing cavities inside the head.

20. Use of a progressive surgical distraction device, according to claim 13, for accessing cavities inside the heart.
